(19) United States
(12) Patent Application Publication
(10) Pub. No.: US 2026/0091326 A1
(43) Pub. Date: Apr. 2, 2026

(54) EXTRACTION SYSTEM AND METHOD

(71) Applicant: SEACHANGE BIOCHEMISTRY INC., Dartmouth, Nova Scotia (CA)

(72) Inventor: Sabrena Mackenzie, Dartmouth, Nova Scotia (CA)

(21) Appl. No.: 19/110,368

(22) PCT Filed: Sep. 8, 2023
(86) PCT No.: PCT/CA2023/051197

§ 371 Date: Mar. 10, 2025

Related U.S. Application Data
(60) Provisional application No. 63/404,784, filed on Sep. 8, 2022.

(51) Int. Cl. B01D 11/02; A61K 36/03

(52) U.S. Cl. CPC B01D 11/0288; A61K 36/03; B01D 11/0257; A61K 2236/15; A61K 2236/37; A61K 2236/53

(57) ABSTRACT

A process for producing a high quality polyphenol-containing antioxidant extract from a seaweed biomass that yields a residual biomass available for alginate and fucoidan production.

This application claims priority to provisional patent application 63/404,784 filed Sep. 8, 2022, the content of which are incorporated herein by reference.

### TECHNICAL FIELD

This disclosure generally relates to the field of obtaining chemicals from a seaweed biomass.

### BACKGROUND OF THE ART

Seaweed is one of the largest under-utilized and valuable resources on the planet. It contains an abundance of nutrients and has applications across many industries, including cosmetics, nutritional products, and biomaterials.

Alginate (alginic acid) is a polysaccharide mainly found in brown algae. There are a number of significant industrial applications for alginates, including as gelling, viscosity and stabilizing agents, with applications in a number of fields, including medical applications, where purity and consistency of product is particularly important. Commercial extraction of alginate is generally by hot alkali extraction, a process that is both environmentally destructive and yields a residual biomass in a state that nothing of further value can be extracted. Most commercial processes also involve a pre-extraction step of soaking the seaweed biomass in formaldehyde to yield a resulting product with less colour.

Polyphenols are a family of naturally occurring organic compounds characterized by multiple phenol units and include flavonoids, phenolic acids and polyphenolic amides. While polyphenols have been used as dyes and for tanning, they are increasingly valued for their health effects and anti-oxidant properties. Polyphenol extraction from seaweed has been performed with ethanol extraction typically at a concentration of at least 20% and as high as 90%-see e.g. CN 10834008, CN108066244 and EP 1977756 A1. Polyphenols extracted using current methods can be unstable and susceptible to extensive oxidation. Particularly, in the context of pharmaceutical and nutraceutical applications, there remains is a need for polyphenol products having high efficacy and stability.

Fucoidan is a long chain sulfated polysaccharide found in various species of brown algae. Fucoidan is considered a high value ingredient used in a range of cosmetic, therapeutic and health care preparations, including dietary supplements, functional foods and dermatological preparations, in which it is valued for anti-oxidant and anti-inflammatory effects among others.

Fucoxanthin ((1S,3R,4M)-3-Hydroxy-4-{(3E,5E,7E,9E,11E,13E,15E,17E)-18-[(1S,4S,6R)-4-hydroxy-2,2,6-trimethyl-7-oxabicyclo[4.1.0]heptan-1-yl]-3,7,12,16-tetramethyl-17-oxooctadeca-1,3,5,7,9,11,13,15,17-nonaen-1-ylidene}-3,5,5-trimethylcyclohexyl acetate) is a xanthophyll, found as an accessory pigment in the chloroplasts of brown algae, giving them a brown or olive-green color. Fucoxanthin is used as a dietary supplement, with evidence of anti-cancer and weight loss and metabolic effects.

### SUMMARY

In accordance with a first embodiment, there is provided process for extracting compounds from a seaweed biomass comprising: performing an antioxidant extraction comprising: an aqueous extraction of the seaweed biomass wherein the aqueous extraction is performed in a solvent comprising ≤10%, preferably between about 1% and about 10%, more preferably between 2.5% and 4.5%, of a C1-C4 alcohol, optionally ethanol or isopropyl alcohol, and wherein the alcohol is used at a volume that is between about 20% and about 40% volume based on the seaweed biomass dry weight, wherein the extraction is performed at a temperature of between about 15° C. and about 60° C. under gentle agitation; separating a crude antioxidant-containing solution from a residual seaweed biomass; and performing ultrafiltration of the crude antioxidant-containing solution to obtain an antioxidant extract containing at least 5% polyphenols by weight, optionally between 5% and 40% polyphenols by weight, between 5% and 35% polyphenols by weight, or between 10%, 15% or 25% and 40% polyphenols by weight.

Other embodiments include:
2. The process of embodiment 1, further comprising drying the antioxidant extract to obtain an antioxidant powder, optionally spray drying or freeze drying the antioxidant extract to obtain the antioxidant powder.
3. The process of embodiment 2, comprising freeze drying the antioxidant extract to obtain the antioxidant powder.
4. The process of embodiment 3, wherein prior to freeze drying, the antioxidant extract is frozen over a period of 12 to 48 hours at a temperature of between about −25° C. and about −5° C.
5. The process of any one of embodiments 1 to 4, wherein the crude antioxidant-containing solution is separated from the residual seaweed biomass by filtration.
6. The process of any one of embodiments 1 to 4, wherein the crude antioxidant-containing solution is separated from the residual seaweed biomass using a centrifugal separator.
7. The process of any one of embodiments 1 to 6, further comprising prior to the antioxidant extraction, extracting fucoxanthin from the seaweed biomass using CO₂ supercritical fluid extraction.
8. The process of any one of embodiments 1 to 7, further comprising prior to or following the antioxidant extraction, preferably prior to the antioxidant extraction, performing a fucoidan extraction using a weak organic acid.
9. The process of any one of embodiments 1 to 8, wherein the seaweed biomass comprises a brown seaweed biomass, preferably from the genus Macrocystis, Laminaria or Ascophyllum, more preferably from Macrocystis pyrifera, Laminaria digitata or Ascophyllum nodosum.
10. The process of any one of embodiments 1 to 9, wherein the seaweed biomass is chopped but not pulverized and dried to a moisture content of ≤about 10%.
11. The process of any one of embodiments 1 to 10, comprising subsequently performing an alginate extraction from the residual biomass, comprising extracting the sodium alginate using a base treatment, preferably wherein the base is Na₂CO₃.
12. The process of embodiment 11, wherein the sodium alginate extraction further comprises prior to extracting from the residual biomass alginate using a base treatment, treating the residual biomass with a dilute acid, preferably HCl or MgCl₂.
13. A process for preparing seaweed from the genus Laminaria, preferably Laminaria digitata, for alginate production comprising separating the stype from the blade and peeling or removing skin from the stype.
14. The process of any one of embodiments 1 to 11, wherein the seaweed biomass comprises the separated blade and the peeled or removed skin of the stype produced by the process of embodiment 13.
15. A seaweed biomass for alginate production comprising the residual biomass produced by the process of any one of embodiments 1 to 10.
16. A seaweed biomass for alginate production, wherein the seaweed is from the genus Laminaria, preferably Laminaria digitata, and the skin of the stype is removed or abraded, preferably wherein <10% of the seaweed biomass comprises the blade of the seaweed.
17. Alginate, preferably sodium alginate, produced by the process of embodiment 11 or 12, from seaweed prepared according to the process of embodiment 13 or from the seaweed biomass of embodiment 15 or 16.
18. A soil ingredient or additive comprising the residual biomass resulting from the process defined in any one of embodiments 1 to 12 or 14. Accordingly, also provided is a process for preparing a soil ingredient or additive comprising obtaining the residual biomass of any one of embodiments 1 to 12 or 14.
19. An antioxidant extract or powder prepared by the process according to any one of embodiments 1 to 12 or 14.
20. A pharmaceutical, nutraceutical or cosmetic product comprising the antioxidant extract or powder of embodiment 19.

Many further features and combinations thereof concerning the present improvements will appear to those skilled in the art following a reading of the instant disclosure.

### DESCRIPTION OF THE DRAWINGS

FIG. 1 is a schematic view of an acid pre-treatment that forms part of an alginate extraction, according to an embodiment.

FIG. 2 is a schematic view of an alginate extraction, according to an embodiment.

FIG. 3 illustrates total antioxidant capacity of Seaweed Extract and Blueberry Extract per Example 2. The data is shown as the average±standard deviation of duplicate data points for each dose. FIG. 3A: The colorimetric readings were used to plot the results onto the gallic acid standard curve and calculate the Gallic Acid Equivalents (GAE). FIG. 3B: The colorimetric readings were used to plot the results onto the phloroglucinol standard curve and calculate the phloroglucinol equivalents.

FIG. 4A illustrates cellular antioxidant protection against AAPH-mediated oxidative stress for Seaweed Extract, Blueberry Extract, Phloroglucinol and Gallic Acid per Example 2. The percent inhibition of cellular oxidative damage was calculated based on the fluorescence readings for cells treated with the test product, compared to negative control cultures (untreated cells) and positive control cultures (treated with the oxidizer AAPH in the absence of antioxidants). The percent inhibition is shown as the average±standard deviation of duplicate data points for each dose.

FIG. 4B illustrates cellular antioxidant protection against H₂O₂-mediated oxidative stress for Seaweed Extract, Blueberry Extract, Phloroglucinol and Gallic Acid per Example 2. The percent inhibition of cellular oxidative damage was calculated based on the fluorescence readings for cells treated with the test product, compared to negative control cultures (untreated cells) and positive control cultures (treated with the oxidizer H₂O₂ in the absence of antioxidants). The percent inhibition is shown as the average±standard deviation of duplicate data points for each dose.

FIG. 5A illustrates cellular energy production under normal culture conditions as a measure of relative mitochondrial metabolic activity in peripheral blood mononuclear cells (PBMC) cultures per Example 2. PBMC were exposed to serial dilutions of products for 24 hours after which time cultures were processed in the colorimetric MTT assay. Results reflect the sum of the metabolic activity of cells in each culture. The colorimetric readings were used to calculate the % change from untreated control cultures, where the solid grey line shows the average of untreated cells, and the lighter grey shade shows the standard deviation for untreated cells. Statistical significance at different doses is indicated by asterisks, when p<0.10: (*), p<0.05: * and p<0.01: **.

FIG. 5B illustrates cellular energy production under oxidative stress conditions as a measure of relative mitochondrial metabolic activity in PBMC cultures per Example 2. PBMC were exposed to serial dilutions of products for 24 hours after which time cultures were processed in the colorimetric MTT assay. Results reflect the sum of the metabolic activity of cells in each culture. The colorimetric readings were used to calculate the % change from cultures treated with H₂O₂, where the solid grey line shows the average of H₂O₂-treated cells, and the lighter grey shade shows the standard deviation for H₂O₂-treated cells. Statistical significance at different doses is indicated by asterisks, when p<0.10: (*), p<0.05: * and p<0.01: **.

FIG. 6A is an image of human dermal fibroblasts cultured under normal un-stressed culture conditions per Example 2. The cells are forming sheets of elongated fibers, each fiber being one skin cell. The Blueberry Extract caused a minor loss of cells. In contrast, the cultures treated with the Seaweed Extract show a thicker and more coherent layer of skin cells.

FIG. 6B is an image human dermal fibroblasts cultured under oxidative stress conditions per Example 2. The skin cells were pre-treated with test products, and then stress was caused by adding H₂O₂ (0.00037%). The stressed cells show disruption of the sheets of elongated fibers, where each skin cell withdraws from its neighbor cell and is unable to maintain a coherent sheet of cells. The Blueberry Extract did not provide much protection from the oxidative stress. In contrast, the cultures treated with the Seaweed Extract immediately prior to adding H₂O₂ showed a thicker and more coherent layer of skin cells, showing a protective effect of the skin cells under oxidative stress.

FIG. 7A illustrates cellular energy production under normal culture conditions as a measure of relative mitochondrial metabolic activity in cultures of primary human dermal fibroblasts per Example 2. Fibroblasts were exposed to serial dilutions of products for 24 hours after which time cultures were processed in the colorimetric MTT assay. Results reflect the sum of the metabolic activity of cells in each culture. The colorimetric readings were used to calculate the % change from untreated control cultures (grey line at 100%). Statistical significance at different doses is indicated by asterisks, when p<0.10: (*), p<0.05: * and p<0.01:

FIG. 7B illustrates cellular energy production under oxidative stress conditions as a measure of relative mitochondrial metabolic activity in cultures of primary human dermal fibroblasts per Example 2. Fibroblasts were exposed to serial dilutions of products, and then stressed with H₂O₂ for 24 hours after which time cultures were processed in the colorimetric MTT assay. Results reflect the sum of the metabolic activity of cells in each culture. The colorimetric readings were used to calculate the % change from cultures treated H₂O₂ (grey line at 100%). Statistical significance at different doses is indicated by asterisks, when p<0.10: (*), p<0.05: * and p<0.01:

FIG. 8. Monocyte numbers: Changes following consumption of test product placebo (B), 50 mg dose of polyphenol seaweed extract powder (L), or 300 mg dose of polyphenol seaweed extract powder (H) per Example 3. FIG. 8A: Cell count/μL blood. FIG. 8B: Percent change from baseline. Average±standard error of the mean is shown for baseline, one hour, and two hours after consumption. Levels of statistical significance are shown in the tables below the graphs where changes from one time point to a later time point after consuming a test product and at the same time point between products is indicated by asterisks, where p<0.10: (*), p<0.05: *, p<0.01: **

FIG. 9. Number of monocytes: Differences in changes following consumption of polyphenol seaweed extract powder versus placebo per Example 3. Average±standard error of the mean is shown for baseline, one hour and two hours after consumption. Levels of statistical significance are shown in the table below the graph where changes from one time point to a later time point is indicated by asterisks, where p<0.10: (*), p<0.05: *, p<0.01: **.

FIG. 10. Lymphocyte numbers: Changes following consumption of test product B, L, or H per Example 3. FIG. 10A: Cell count/μL blood. FIG. 10B: Percent change from baseline. Average±standard error of the mean is shown for baseline, one hour, and two hours after consumption. Levels of statistical significance are shown in the tables below the graphs where changes from one time point to a later time point after consuming a test product and at the same time point between products is indicated by asterisks, where p<0.10: (*), p<0.05: *, p<0.01: **.

FIG. 11. Number of lymphocytes: Differences in changes following consumption of polyphenol seaweed extract powder versus placebo per Example 3. Average±standard error of the mean is shown for baseline, one hour and two hours after consumption. Levels of statistical significance are shown in the table below the graph where changes from one time point to a later time point is indicated by asterisks, where p<0.10: (*), p<0.05: *, p<0.01: **.

FIG. 12. NK numbers: Changes following consumption of test product B, L, or H per Example 3. FIG. 12A: Cell count/μL blood. FIG. 12B: Percent change from baseline. Average±standard error of the mean is shown for baseline, one hour, and two hours after consumption. Levels of statistical significance are shown in the tables below the graphs where changes from one time point to a later time point after consuming a test product and at the same time point between products is indicated by asterisks, where p<0.10: (*), p<0.05: *, p<0.01: **.

FIG. 13. Number of NK cells: Differences in changes following consumption of polyphenol seaweed extract powder versus placebo per Example 3. Average±standard error of the mean is shown for baseline, one hour and two hours after consumption. Levels of statistical significance are shown in the table below the graph where changes from one time point to a later time point is indicated by asterisks, where p<0.10: (*), p<0.05: *, p<0.01: **.

FIG. 14. T numbers: Changes following consumption of test product B, L, or H per Example 3. FIG. 14A: Cell count/μL blood. FIG. 14B: Percent change from baseline. Average±standard error of the mean is shown for baseline, one hour, and two hours after consumption. Levels of statistical significance are shown in the tables below the graphs where changes from one time point to a later time point after consuming a test product and at the same time point between products is indicated by asterisks, where p<0.10: (*), p<0.05: *, p<0.01: **.

FIG. 15. Number of T cells: Differences in changes following consumption of polyphenol seaweed extract powder versus placebo per Example 3. Average±standard error of the mean is shown for baseline, one hour and two hours after consumption. Levels of statistical significance are shown in the table below the graph where changes from one time point to a later time point is indicated by asterisks, where p<0.10: (*), p<0.05: * p<0.01: **.

FIG. 16. nonNKnonT numbers: Changes following consumption of test product B, L, or H per Example 3. FIG. 16A: Cell count/μL blood. FIG. 16B: Percent change from baseline. Average±standard error of the mean is shown for baseline, one hour, and two hours after consumption. Levels of statistical significance are shown in the tables below the graphs where changes from one time point to a later time point after consuming a test product and at the same time point between products is indicated by asterisks, where p<0.10: (*), p<0.05: *, p<0.01: **.

FIG. 17. Number of non-NK non-T cells: Differences in changes following consumption of polyphenol seaweed extract powder versus placebo per Example 3. Average±standard error of the mean is shown for baseline, one hour and two hours after consumption. Levels of statistical significance are shown in the table below the graph where changes from one time point to a later time point is indicated by asterisks, where p<0.10: (*), p<0.05: *, p<0.01: **.

FIG. 18. Number of CD45-CD31+CD309+ endothelial stem cells: Changes following consumption of B versus L versus H per Example 3. FIG. 18A: Cell count/μL blood. FIG. 18B: Percent change from baseline. Average±standard error of the mean is shown for baseline, one hour, two hours and one week after consumption. Levels of statistical significance are shown in the table below the graphs where changes from one time point to a later time point after consuming a test product and at the same time point between products is indicated by asterisks, where p<0.10: (*), p<0.05: *, p<0.01: **.

FIG. 19. Number of CD45-CD31+CD309+ endothelial stem cells Differences in changes following consumption of polyphenol seaweed extract powder versus placebo per Example 3. Average±standard error of the mean is shown for baseline, one hour and two hours after consumption. Levels of statistical significance are shown in the table below the graph where changes from one time point to a later time point is indicated by asterisks, where p<0.10: (*), p<0.05: *, p<0.01: **.

FIG. 20. Number of CD45-CD90++ lymphocytes: Changes following consumption of B versus L versus H per Example 3. FIG. 20A: Cell count/μL blood. FIG. 20B: Percent change from baseline. Average±standard error of the mean is shown for baseline, one hour, two hours and one week after consumption. Levels of statistical significance are shown in the table below the graphs where changes from one time point to a later time point after consuming a test product and at the same time point between products is indicated by asterisks, where p<0.10: (*), p<0.05: *, p<0.01: **.

FIG. 21. Number of CD45-CD90++ lymphocytes: Differences in changes following consumption of polyphenol seaweed extract powder versus placebo per Example 3. Average±standard error of the mean is shown for baseline, one hour and two hours after consumption. Levels of statistical significance are shown in the table below the graph where changes from one time point to a later time point is indicated by asterisks, where p<0.10: (*), p<0.05: *, p<0.01: **.

### DETAILED DESCRIPTION

In one embodiment, there is provided a novel process for obtaining an antioxidant extract comprising polyphenols from a seaweed biomass and, in a preferred embodiment, brown algae. This novel process yields a polyphenol product of high quality and having enhanced antioxidant activity and immune support compared to products produced by known processes. This process further yields a residual seaweed biomass that is suitable for alginate and/or fucoidan production. In one embodiment, there is provided a starting seaweed biomass suitable for alginate production having a low polyphenol content and that has not been exposed to formaldehyde. In some embodiment, fucoxanthin and/or fucoidan is obtained from the seaweed biomass prior to polyphenol production.

As used herein, “alginate” includes alginic acid and alginate salts (e.g. calcium salt, sodium salt, magnesium salt), and can include derivatives of alginic acid, such as esters.

As used herein, an “antioxidant extract” is a seaweed extract comprising at least 5% e.g. between 5% and 40% of compounds having antioxidant effects in cells and in a particular embodiment between 5% and 30% polyphenols.

As used herein, “polyphenols” include naturally occurring organic compounds extracted or extractable from seaweed characterized by multiple phenol units and includes flavonoids, phenolic acids and polyphenolic amides, and notably phlorotannins, which are the predominant polyphenols in brown seaweed. Polyphenol content measured using the Folin-Ciocalteu assay

Although terms such as “maximize”, “minimize” and “optimize” may be used in the present disclosure, it should be understood that such term may be used to refer to improvements, tuning and refinements which may not be strictly limited to maximal, minimal or optimal.

The term “substantially” as used herein may be applied to modify any quantitative representation which could permissibly vary without resulting in a change in the basic function to which it is related.

Terms such as “up to”, “at least”, “greater than”, “less than”, “more than”, “or more”, and the like, include the number recited and such terms refer to ranges that can be subsequently broken down into sub-ranges. In the same manner, all ratios recited herein also include all sub-ratios falling within the broader ratio.

The singular forms “a,” “an,” and “the” include the plural reference unless the context clearly dictates otherwise. The term “and/or” means any one of the items, any combination of the items, or all of the items with which this term is associated.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

In broadest embodiments, any seaweed species that is a source of polyphenols can be used in the process provided herein. Preferably, the seaweed biomass is a brown seaweed biomass. Genera of brown seaweed (Phaeophyceae) include Fucus, Laminaria, Macrocystis and Sargassum. As will be apparent to a person of skill in the art, the seaweed biomass may be composed of a single species or be composed of a mixture of species.

In preferred embodiments, the brown algae is from the genus Macrocystis, Laminaria or Ascophyllum. In some embodiments, the brown algae is Macrocystis pyrifera or Laminaria digitata. In one embodiment, Ascophyllum nodosum.

The seaweed biomass used in the process described below is suitably chopped, but not pulverized, and dried under controlled condition, in preferred embodiments to a moisture content of ≤10%.

### Antioxidant Extract

An antioxidant extract containing significant levels of polyphenols is obtained by aqueous extraction of seaweed without significant heating. The extraction medium includes (in some embodiments consists or consists substantially) water and a C1-C4 alcohol, preferably ethanol or isopropyl alcohol. The alcohol in the aqueous extraction medium is ≤10%, more preferably ≤5%, in one embodiment, between about 1% and about 5%, preferably between about 2% and about 4%. Further, the alcohol is used at a volume that is between about 20% and about 40% of the initial (i.e. dry, ≤10% moisture) seaweed weight, more preferably between about 27% and about 33% volume based on the initial seaweed weight. The extraction is performed at a temperature of between 15° C. and 60° C., more preferably between 20° and 30° C., most preferably at room temperature i.e. about 22° C. to about 25° C. The extraction is suitably performed under conditions of gentle agitation without centrifugation or aggressive mixing. Suitably, the level of agitation is sufficiently gentle that the integrity of the algal cell walls remain substantially intact. While reference to gentle agitation indicates that aggressive techniques are avoided, it will be understood by a person of skill in the art that the gentle agitation may be constant or intermittent and different techniques of (gentle) agitation may be used during an extraction process The seaweed biomass is suitably agitated in the extraction medium for a period of between about 2 and about 8 hours, more preferably about 3-5 hours. The extraction may be performed twice on the same starting biomass.

Following solvent extraction, a crude antioxidant solution is separated from the residual seaweed biomass. In various embodiments, the residual seaweed biomass may be separated using any suitable non-destructive method known to those of skill in the art e.g. filtration, centrifugation. In one embodiment, the seaweed residual biomass is separated using a screen and the crude antioxidant solution is filtered. In another embodiment, a centrifugal separator is used.

The crude antioxidant solution is subject to ultrafiltration to remove residual contaminating chemicals such as heavy metals, iodine and arsenic. In some embodiments, the ultrafiltration step decreases the level of one or more contaminating chemicals, in some embodiments to below detectable levels. Suitably, the filter size is between about 2500 Daltons and about 5500 Daltons, more preferably between about 3000 Daltons and about 5000 Daltons. As water (containing the contaminating chemicals) is removed through ultrafiltration, the retentate extract is concentrated.

The (ultrafiltered) antioxidant solution is suitably dried to obtain a powder. In some embodiments, drying may be by any conventional method e.g. spray drying. In a preferred embodiment, a freeze dryer is used, as the present inventors have found that drying under vacuum (i.e. in the absence of air) eliminates oxidation, further maintaining the antioxidant activity of the polyphenol product. In a preferred embodiment, prior to freeze drying, the antioxidant solution is subject to a slow freezing step, suitably the solution is frozen over a period of 12-48 hours at a temperature of between about −25° C. and −5° C., in some embodiments at a temperature of between about −20° C. and −10° C. It has been determined that this slow freezing step improves the polyphenol content of the extract as compared to immediately freeze drying the solution. In some embodiments, the drying step yields a powder suitably having a moisture content of <10%, in some embodiments between about 5% and about 7% by weight.

The extraction process provides commercially useful yields, in some embodiments an extract yield of between 5 and 20%, in one embodiment, about 10% based on the weight of the initial seaweed biomass.

As detailed in the Examples, the antioxidant extract as provided herein has improved stability over known products and consequently increased bioavailability and antioxidant capacity. Further, the disclosed methods are more environmentally friendly than prior methods and, in particular, can avoid the use of formaldehyde. The disclosed method also yields a consistent product suitable for nutraceutical and cosmetic applications.

### Alginate Production

The residual biomass from the antioxidant extraction described above can be subject to alginate extraction to yield a high quality alginate product. The process provided herein is particularly advantageous because the antioxidant extraction step (which as described above is consistent with the principles of green chemistry) yields a starting biomass for alginate extraction that does not require a formaldehyde treatment to reduce colour. The antioxidant extraction step itself reduces the alginate colour, which can be further reduced (evidencing a highly pure product) by treatment with a dilute acid, which does not have the environmental or toxicity concerns of the formaldehyde treatment standard in the industry. In another embodiment, the alginate colour may be reduced using a physical means, which is particularly suitable for species from the genus Laminaria, including the species Laminaria digitata. Under this method, the stype and blade are separate and the polyphenol-rich outer skin of the stype is removed by physical means, for example, peeling with a blade or abrading the skin. Alginate refined from the stripped stype exhibits a light colour and a consistent high quality fiber, while the separated skin portions are suitably directed to polyphenol extract production. In some refining processes, multiple colour reduction methods may be employed.

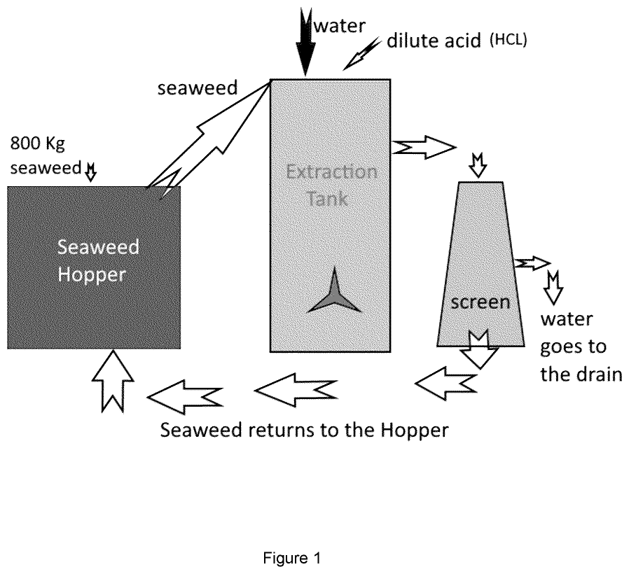

With reference to FIG. 1, in one embodiment, the alginate extraction comprises an acid pre-extraction treatment to remove any residual phenol. This acid treatment is suitably performed with a dilute acid (e.g. HCl or MgCl₂), suitably with draining. Suitably, the acid pre-extraction treatment is performed at a temperature of between about 15° C. and 60° C., more preferably between about 20° C. and 40° C.

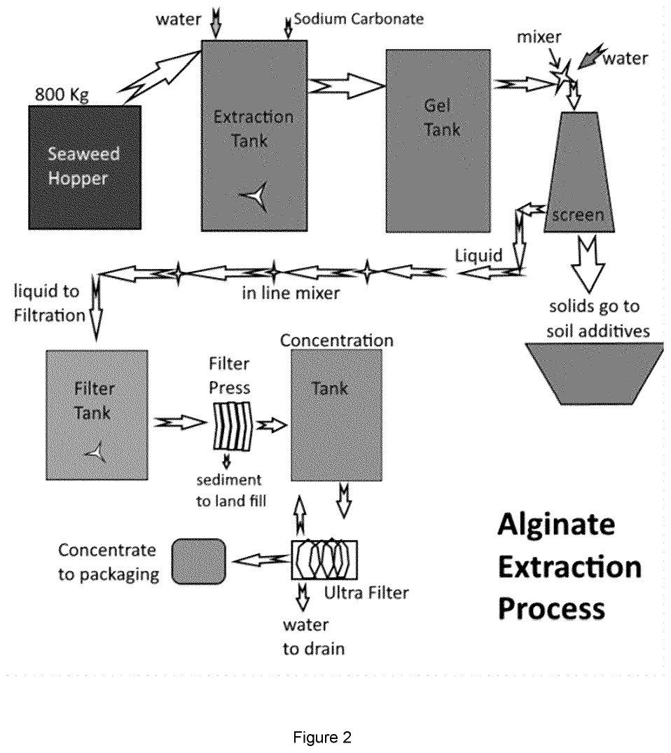

With reference to FIG. 2, the alginate extraction as provided herein comprises base treatment, suitably with Na₂CO₃ of the brown seaweed solid to solubilize sodium alginate. Following base treatment, solid particles are separated from the sodium alginate solution. The separation step may be performed using any method known to those of skill in the art, such as centrifugation or filtration.

Suitably, the alginate extract is concentrated using ultrafiltration. In some embodiments, using a filter size of between about 2500 and about 10000 Daltons, more preferably between about 5000 Daltons and about 10000 Daltons.

Optionally, the sodium alginate may be dried using methods known in the art, such as air drying, oven drying, freeze drying or drying under vacuum. Optionally, the sodium alginate solution may be extruded to provide e.g. sodium alginate pellets. Extruded sodium alginate pellets can suitably be dried and milled according to processes known in the art.

The earlier antioxidant extraction step in combination with the process provided herein yields a high quality alginate product

### Fucoidan Production

In one embodiment, prior to alginate extraction, a fucoidan extraction is performed from the residual seaweed biomass resulting from the antioxidant extraction. The antioxidant extraction process provided herein yields a biomass from which a high quality fucoidan product can be extracted and, further, the fucoidan extraction process is non-destructive in that the resulting biomass is susceptible to alginate extraction. In other embodiments, fucoidan is extracted prior to antioxidant extraction. The fucoidan extraction is suitably performed in the presence of an aqueous solvent comprising a weak organic acid, in one embodiment, a combination of at least two or two weak organic acids is used. In some embodiments, the extraction medium comprises one or more of citric acid, acetic acid, and malic acid. The fucoidan extraction is performed at a temperature between 15° C. and 60° C., more preferably between 20° and 30° C., suitably under gentle agitation.

### Fucoxanthin Production

In one embodiment, prior to antioxidant extraction, a fucoxanthin extraction is performed on the seaweed biomass. Fucoxanthin extraction may be performed using supercritical fluid extraction, suitably using CO₂. Examples of fucoxanthin extraction from seaweed can be found in A. T. Quitain et al. Supercritical carbon dioxide extraction of fucoxanthin from Undaria pinnatifida, J Agric Food Chem. 2013 Jun. 19; 61 (24); 5792-7; and S. P. Sivagnanam et al. Biological Properties of Fucoxanthin in Oil Recovered from Two Brown Seaweeds Using Supercritical CO2 Extraction, Mar Drugs. 2015 May 29; 13 (6): 3422-42. Because supercritical fluid extraction can be performed at low temperature, it avoids destruction of the polyphenols and alginate enabling the subsequent process steps. Extracting fucoxanthin from the biomass prior to alginate production further contributes to the decolourization of the alginate product thereby dispensing with the need for formaldehyde treatment.

### Soil Ingredient or Additive

The residual biomass of the above described process (i.e. comprising polyphenol and alginate extraction and, optionally, fucoidan and/or fucoxanthin extraction) has a high phosphorus content and is suitable for use as a soil ingredient or additive. This soil ingredient or additive has a unique composition over known soil ingredients or additives.

### Process

Suitably, all steps prior to alginate production are performed under conditions that avoid temperatures in excess of about 60° C., preferably in excess of about 40° C. and avoid pulverizing the seaweed biomass so that the integrity of the majority of algal cell walls is substantially maintained.

The examples described above and illustrated are intended to be exemplary only. The scope is indicated by the appended claims. However, it should be understood that the disclosure includes the specific production examples provided in the Examples with any of the specific parameters understood to be modified by the term “about”.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

### EXAMPLES

### Example 1: Antioxidant Extract Production

In this example 500 Kg Seaweed (initial dry seaweed weight) is used, however, batch sizes may vary according to the production needs. This recipe uses percentage of the initial dry seaweed weight, as a means of covering various amounts of seaweed extracted.

Before each batch, all tanks and lines are given a thorough cleaning and sanitizing. This is a cold extraction. Normally no heat is added to the process. When necessary heat may be added to bring the temperature up to room temperature (22°-25° C.).

Water is first added to an Extraction Tank. The volume is calculated as 10× the seaweed weight, i.e. for 500 Kg seaweed, 5000 L water is added to the tank. Ethanol is added next at a volume that is 35% of the seaweed weight, i.e. for 500 Kg this works out to 175 L.

Gentle agitation is begun and the seaweed is added. A pH adjustment is performed on the first extraction. An amount of 10% Soda Ash solution is added to the extraction tank to raise the pH to 7.0-7.5. After 50 minutes, 10% HCL solution is added to bring the pH down to 6.5-7.0. This pH manipulation is only done on the first extraction. Agitation continues for 3 more hours, for a total of four hours.

The Slurry is then pumped over an 80-mesh dewatering screen to separate the solids from the liquids. Alternatively, a centrifugal separator is used to separate solids from the liquids. The seaweed is returned to the hopper and can be subject to a second extraction. The separated liquid is transferred to a filtering tank.

Activated carbon powder is added to the tank as it begins to fill. The amount is calculated as 0.5% of the seaweed weight i.e. for 500 Kg, 2.5 Kg Carbon. The mixture is agitated to keep the solids in suspension. Once the filter tank contains all the liquor, Filter Aid (diatomaceous earth) is added to the tank. The amount is calculated as 7% of the seaweed weight, i.e. for 500 Kg, 35 Kg of filter aid. The Filter press is coated with a thin layer of filter aid. A clean, 1 micron filter bag is placed in the canister after the filter press, as a failsafe, and pressure filtering begins. The clean liquor moves on to the Concentration Tank.

While the liquor is being filtered, the second extraction can begin. The volume of water is reduced to 8 to 1 to allow for the absorption of water by the seaweed in the first extraction. This time the amount of alcohol is calculated as 30% of the dry seaweed weight i.e. for 500 Kg, 4000 L water and 150 L Ethanol. The second extraction is gently agitated in the extraction medium for a total of 3 hours.

Concentration is performed with an ultrafilter. The filter is sized to separate water, salts and unwanted heavy metals while holding back the antioxidant extract. A size of 5000 Daltons is suitable.

When the liquor begins to fill the Concentration Tank, the Ultra Filter is turned on and concentration begins. The volume of liquid decreases as water is removed and the extract becomes more concentrated.

The liquor from the second extraction is similarly processed i.e. activated Carbon powder and Filter Aid as before. The clean liquor is added to the concentration tank so that both extractions are mixed by the circulation in this tank. The two extractions combine to form one, uniform, batch of product.

The liquid is concentrated down in volume via ultrafiltration until a solids content of 10-12%, is reached and transferred to a Finishing tank.

The concentrated liquor is transferred to a freeze dryer.

The dry product can be ground and an 80 mesh sieve may be used to create a uniform powder.

### Example 2: Novel Seaweed Extract: Antioxidant Protection at the Cellular Level

A polyphenol seaweed extract powder prepared according to polyphenol extraction methods described herein was compared to a best in class blueberry extract powder. On the morning of each laboratory testing day, a fresh stock solution was prepared in physiological saline at 100 mg/mL. The powders were allowed to hydrate under gentle agitation, after which any remaining solids were removed by centrifugation, followed by filtration through 0.22-micron cellulose acetate syringe filters. Serial dilutions were prepared in distilled water for the Folin-Ciocalteu assay, and in phosphate-buffered saline for all other tests.

The two products were tested and compared in a selected panel of lab assays focused on antioxidant properties and cellular protection under normal and stressed culture conditions. In particular, the following tests were performed:

### Total Antioxidant Capacity, Using the Folin-Ciocalteu Assay

The products were tested in the Folin-Ciocalteu assay (also known as the total phenolics assay). This assay makes use of the Folin-Ciocalteu reagent to measure antioxidants. The assay is performed by adding the Folin-Ciocalteu's phenol reagent to serial dilutions of extract, thoroughly mixing, and incubating for 5 minutes. Sodium carbonate is added, starting a chemical reaction producing a color. The reaction is allowed to continue for up to 30 minutes at 37° C. Optical absorbance is measured at 765 nm in a colorimetric plate reader. Gallic acid is used as a reference standard, and data reported in Gallic Acid Equivalents per gram product.

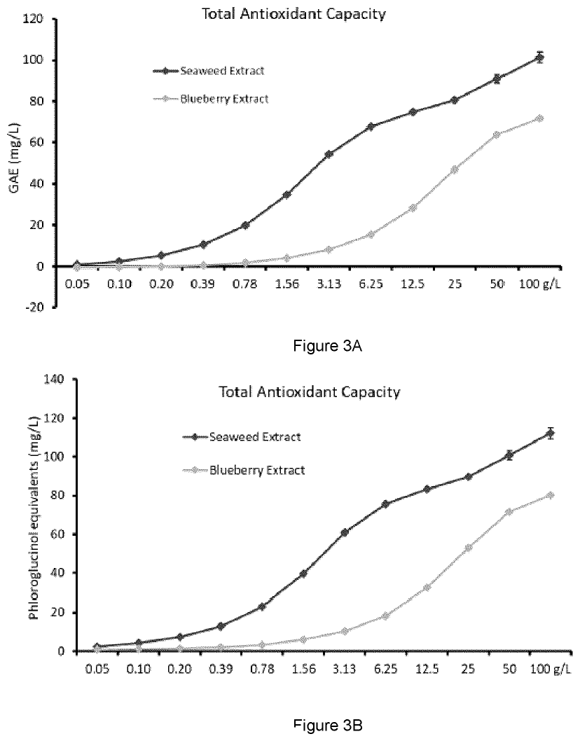

Both extracts showed antioxidant capacity in the Folin-Ciocalteu assay; however, the Seaweed Extract showed more robust antioxidant capacity than the Blueberry Extract. The data were plotted against a standard curve using gallic acid. The data were also plotted against a standard curve using phloroglucinol—see FIGS. 3a and 3b.

TABLE 1
Total antioxidant capacity

| | mg phloroglucinol | mg GAE per |
|---|---|---|
| Product | equivalents per gram product | gram product |
| Seaweed Extract | 25 | 22 |
| Blueberry Extract | 3 | 2 |

### Cell-Based Antioxidant Protection (CAP-e) Assay

The Cell-based Antioxidant Protection (CAP-e) assay allows assessment of anti-oxidant potential in a method that is comparable to the ORAC test, but only allows measurement of antioxidants that are able to cross the lipid bilayer cell membrane, enter the cells, and provide biologically meaningful antioxidant protection under conditions of oxidative stress.

Human red blood cells (RBCs) were washed repeatedly in physiological saline, and then exposed to the test products. During the incubation with a test product, any antioxidant compounds able to cross the cell membrane can enter the interior of the RBC. Then the RBC were washed to remove compounds that were not absorbed by the cells, and loaded with the DCF-DA dye, which turns fluorescent upon exposure to reactive oxygen species.

Oxidation was triggered by addition of the peroxyl free radical generator AAPH and H₂O₂. Therefore, the antioxidant properties of compounds in the novel seaweed extracts were tested in context of 2 different free radical generators: AAPH and H₂O₂. Algal polyphenols have a different chemical composition than land-based plants. Due to the chemical differences the electron donation properties may differ, and this is the reason for exploring the antioxidant properties in context of both types of stressors.

The fluorescence intensity was evaluated. The low fluorescence intensity of untreated control cells served as a baseline, and RBC treated with AAPH alone served as a positive control for maximum oxidative damage.

Observing a reduced fluorescence intensity of RBC exposed to a test product and subsequently exposed to AAPH, indicates that the test product contains antioxidants available to penetrate into the cells and protect these from oxidative damage. Based on the low fluorescence of the untreated control wells, and the high fluorescence of the cell cultures exposed to oxidative damage, the fluorescence intensity in cell cultures treated with test products prior to exposure to oxidative stress was used to calculate the percent inhibition of cellular oxidative stress.

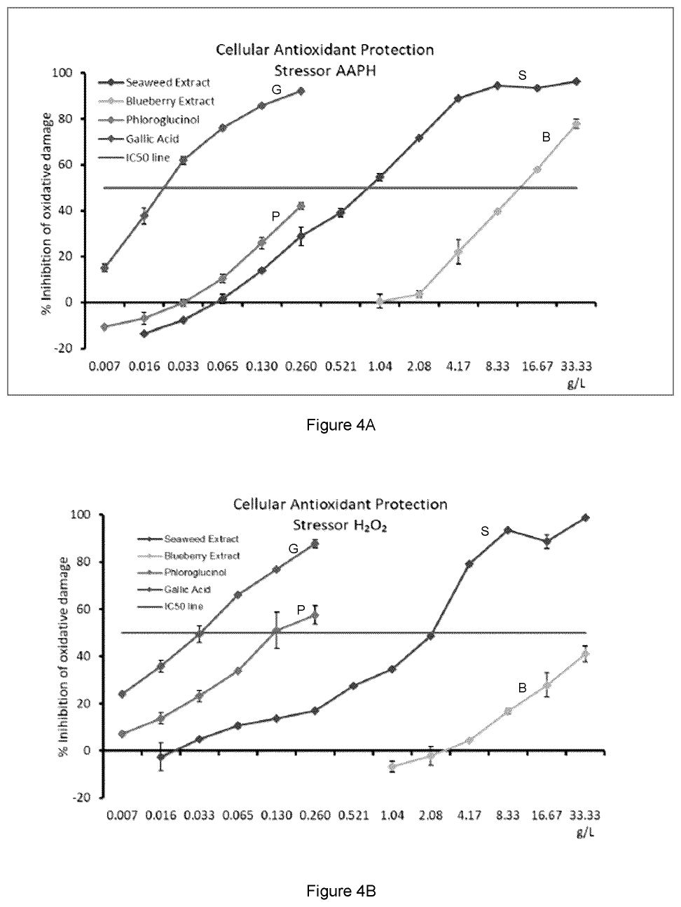

Both extracts showed cellular antioxidant protection in the CAP-e bioassay, however, the Seaweed Extract showed more robust cellular antioxidant protection than the Blueberry Extract, where less Seaweed Extract was needed to inhibit oxidative stress (FIG. 4). The extracts were also compared to gallic acid and phloroglucinol, and the cellular antioxidant protection by the Seaweed Extract was almost as potent as pure phloroglucinol (FIG. 4).

The Seaweed Extract provided protection against both oxidizing agents AAPH and H₂O₂ (Table 2).

TABLE 2
Cellular Antioxidant Protection

| Product | Stressor | μM GA/gram |
|---|---|---|
| Seaweed Extract | AAPH | 142 |
| Blueberry Extract | AAPH | 10 |
| Seaweed Extract | H₂O₂ | 80 |
| Blueberry Extract | H₂O₂ | N/A* |

*Cellular antioxidant protection was evident but the level of protection did not reach 50% (IC50). Since an IC50 value was not achieved, a CAP-e value could not be calculated.

### Mitochondrial Metabolic Activity as an Indicator of Cellular Viability

Peripheral blood mononuclear cells (PBMC) were tested for mitochondrial function using the MTT assay. The MTT assay utilizes a dye that changes color dependent on mitochondrial metabolic activity. Freshly harvested cells were cultured to allow the color formation to take place in proportion to mitochondrial function. In the MTT bioassay, chemical reactions trigger a specific color development based on cellular functions. When a reduction in color is measured, this is linked to a reduced cellular viability, either as a result of direct killing, or inhibition of mitochondrial function. When an increase in color is measured, this has several possible explanations: 1) increased cell numbers (growth); 2) increased mitochondrial mass, and 3) increased mitochondrial function (energy production).

Under the test conditions used, no cell division was expected during the 24-hour cultures. Therefore, an increase in color is proportional to increased mitochondrial function.

The MTT assay was conducted under 2 culture conditions in parallel:
Normal culture conditions—This examined a direct effect of mitochondrial support by compounds able to enter into living cells and interact in ongoing redox reactions.
Oxidative stress culture conditions—This examined the protective effects of the seaweed extracts when cells were stressed with H₂O₂.

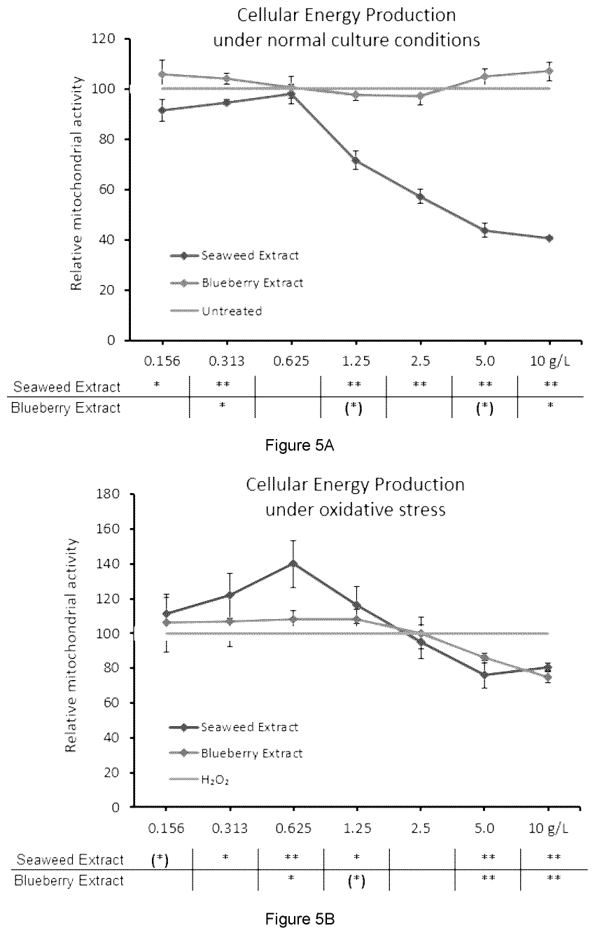

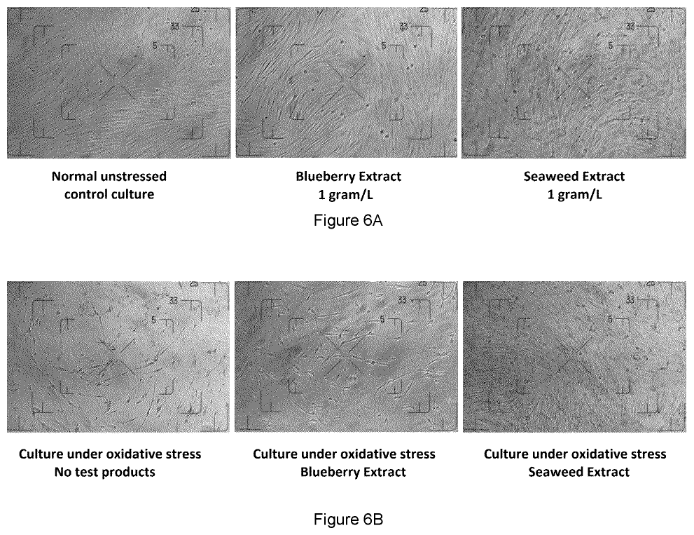

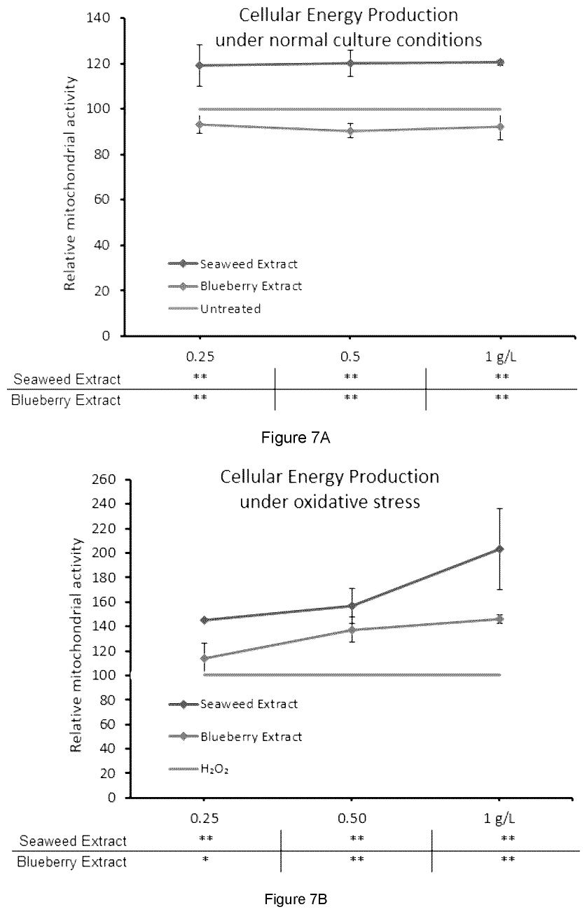

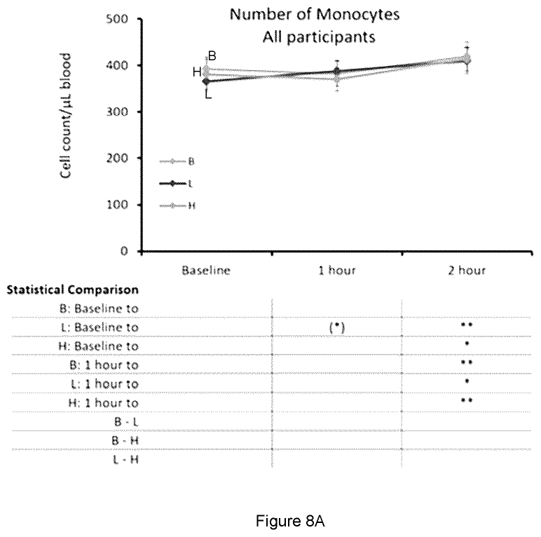

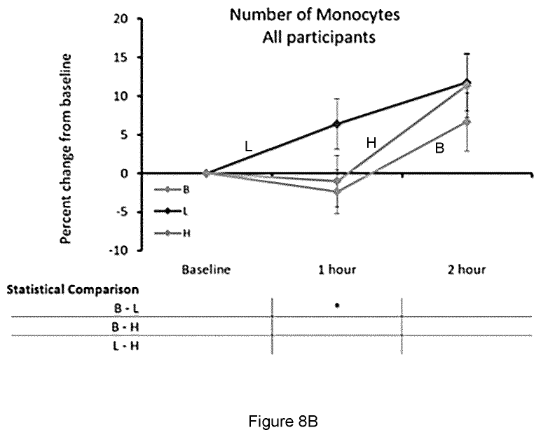

With reference to FIG. 5A, under normal, un-stressed culture conditions, at the 4 higher doses the Seaweed Extract caused some metabolic stress to the cells, while at the 3 lower doses the Seaweed Extract was tolerated by the cells. The Blueberry Extract was tolerated by the cells at all 7 doses tested.

With reference to FIG. 5B, under H₂O₂-induced oxidative stress conditions, at the 2 higher doses the Seaweed Extract and the Blueberry Extract caused some metabolic stress to the cells, while at lower doses, the Seaweed Extract showed protection of the cellular metabolic activity against oxidative stress. At the 3rd lowest dose this protection reached a high level of statistical significance when compared to H₂O₂-treated control cultures. The Blueberry Extract did not provide protection of the cellular metabolic activity against oxidative stress.

The data generated from this testing helped identify the optimal dose range for the following testing on human dermal fibroblasts

### Human Dermal Fibroblasts

Human dermal fibroblast cells were tested for cell viability through testing of mitochondrial function using the MTT assay as described above for peripheral blood mononuclear cells. H₂O₂ was used as the stressor. The testing under normal culture conditions helped demonstrate direct effects on skin cells, and the cultures under the H₂O₂-mediated stress provided evidence as to whether the test products helped reduce oxidative stress to the skin cells, of relevance for stress associated with UV radiation where H₂O₂ is one of the free radicals formed. The doses of the 2 test products were chosen based on the data from testing of effects on PBMC.

Under normal, unstressed culture conditions, the Seaweed Extract supported the growth and formation of a coherent layer of skin cells. This was highly significant at all 3 doses. The Blueberry Extract triggered a very mild reduction of cell growth. This was highly significant at all 3 doses.

Under H₂O₂-induced oxidative stress conditions, the dermal fibroblasts showed dramatically reduced growth and viability, the cells shriveled up, and did not form a uniform layer. The Blueberry Extract provided some protection of the cells, and the cells were less shriveled. Each live cell looked less stressed. However, the growth was still much reduced, compared to healthy unstressed cultures. The protection of cellular metabolic activity was highly significant at the 2 higher doses of product. The Seaweed Extract showed protection of the cellular metabolic activity against oxidative stress. At the highest dose, the layer was almost coherent although not quite as healthy as unstressed cultures of dermal fibroblasts. The protection of cellular metabolic activity was highly significant at all 3 doses of product.

### Example 3: In Vivo Study on Rapid Effects on Immune Alertness and Regenerative Activity of Novel Seaweed Extract

A clinical study was performed to investigate the effect of polyphenol seaweed extract powder prepared according to polyphenol extraction methods described herein on immune alertness and regenerative activity.

A randomized double-blind placebo-controlled cross-over study design of 15 healthy participants was used, where each study participant attended three clinic visits, and consumed 0 mg (Placebo), 50 mg, and 300 mg of the polyphenol seaweed extract on different visits in random order. The study design has been used in previous clinical studies on immune modulating products (see references 1-4). The test parameters evaluated do not necessarily stay constant, even over a few hours, since they are related to people's metabolism, individual circadian rhythms, and other normal physiological parameters. Therefore, the study design included a placebo test day, allowing within-subject analysis of changes between the test days for each person.

Prior to the clinical phase, multiple samples of the polyphenol seaweed extract were prepared into 50 mL Falcon tubes, each containing either 0 mg, 50 mg, or 300 mg of the powder. The tubes were organized according to the randomization scheme, ensuring that each participant would consume 0 mg on one day, 50 mg on another day, and 300 mg on another day. It was also ensured that on any given clinic day, more than one type of test product would be consumed, to avoid for example all participants on one day would consume 0 mg. 45 mL plain rice milk was added to each tube, and tubes containing the polyphenol seaweed extract powder were gently agitated until the powder was completely dissolved into the rice milk.

On each clinic day, immediately after the baseline blood draw, study participants were given a tube to consume in the presence of the clinic staff. Study participants consumed the contents of their allocated tube with water and a few bland soda crackers to stimulate digestive function.

The study of acute changes to levels, activation status, and functionality of immune cells is not trivial. All test parameters undergo circadian changes, and are negatively affected by stress, adrenaline, lack of sleep, and recent illness. The study participants were instructed to call and reschedule a certain clinic day if they felt that any of these things are reasons to do so. The study environment was kept controlled for stressors.

Increases versus decreases in numbers of immune cells in the blood is a measure of cellular trafficking in and out of the blood stream. Any systematic changes observed in a majority of the study participants after consuming the same test product suggests immune activating events are induced. Another aspect of signals that can occur rapidly after consuming a natural restorative and anti-ageing nutraceutical product is related to stem cell trafficking, i.e., the recruitment and mobilization of stem cells as they seek tissue in need of repair, measurable by changes to the numbers of different types of stem cells in the blood circulation.

Freshly drawn blood samples were used for testing changes in immune cell numbers. The cells from each blood draw were assayed in triplicate.

Cells were stained with the T cell marker CD3 and the CD56 marker, as well as the two activation markers CD69 and the interleukin-2 receptor CD25. This allowed analysis of numbers of the following types of immune cells in the blood circulation at each time point in the study:
Monocyte numbers;
Lymphocyte numbers;
NK cells: CD3-negative, CD56-positive lymphocytes;
NKT cells: CD3+CD56+ lymphocytes;
T lymphocytes: CD3+CD56-lymphocytes;
Non-NK, non-T lymphocytes: CD3-CD56-lymphocytes.

The stained cells were analyzed by multi-parameter flow cytometry, using an acoustic dual laser Attune flow cytometer. During data analysis, the physical properties of different cell types allowed electronic gating on lymphocytes versus monocytes, so that the CD69 versus CD25 expression could be analyzed on these cell types separately. In addition, the lymphocyte fraction was divided into 4 separate subpopulations, based on whether cells stain with CD3, CD56, both, or none.

All blood samples were tested in triplicate for the flow cytometric evaluation of immune and stem cells. The triplicates were averaged, and the changes over time were evaluated. For each data set, 3 data graphs were generated:
1. Raw data: Group averages from all baseline samples, 1-hour samples and 2-hour samples after consuming placebo, low dose of polyphenol seaweed extract powder, and the high dose of polyphenol seaweed extract powder.
2. Percent change from baseline: The group average at baseline is set to “0”, and the percent change from baseline is plotted for each consumable product.
3. Difference in percent change: This graph shows the change after consuming a test product, where the change after consuming placebo is subtracted. This data typically gives the best statistical evaluation. Therefore, when levels of statistical significance is mentioned in the text, it is referring to statistics from this calculated value.

Freshly drawn blood samples were used for testing changes in stem cell numbers and phenotype. Rapid changes to the numbers of stem cells in the blood circulation suggest that consumption of a test product transmits a signal to stem cells to enhance the surveillance of the body for tissue in need of repair and rejuvenation. The cells from each blood draw were assayed in triplicate. The following markers were used: CD45, CD34, CD31, CD309 (KDR), and CD90. Data analysis was performed to examine changes in stem cell numbers within each distinct phenotype: CD45dim CD34+ stem cells, as well as CD45−CD31+CD309 (KDR)+ endothelial stem cells. The inclusion of CD90 in the panel allowed analysis for CD45−CD90+ mesenchymal stem cells.

This allowed analysis of numbers of the following types of stem cells in the blood circulation at each time point in the study:
CD34+CD45dim classical stem cells, divided into 2 subsets:
   CD34+CD309+ pluripotential stem cells;
   CD34+CD309− progenitor cells;
CD45− CD31+CD309 (KDR)+ endothelial stem cells;
CD45− CD90++ lymphocytoid stem cells;
Very Small Embryonic-Like Stem Cells (VSELS).

### Cell Numbers in Blood Circulation

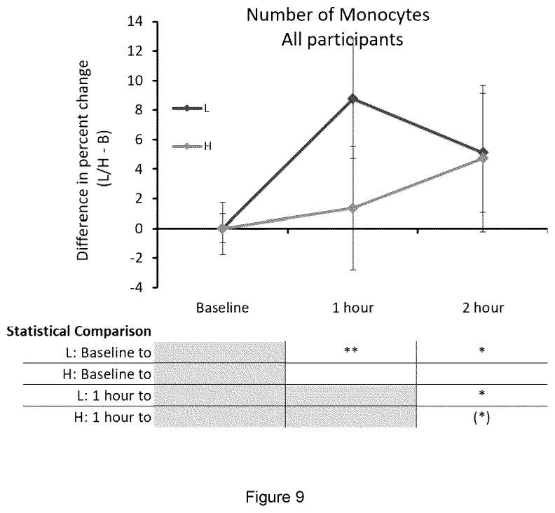

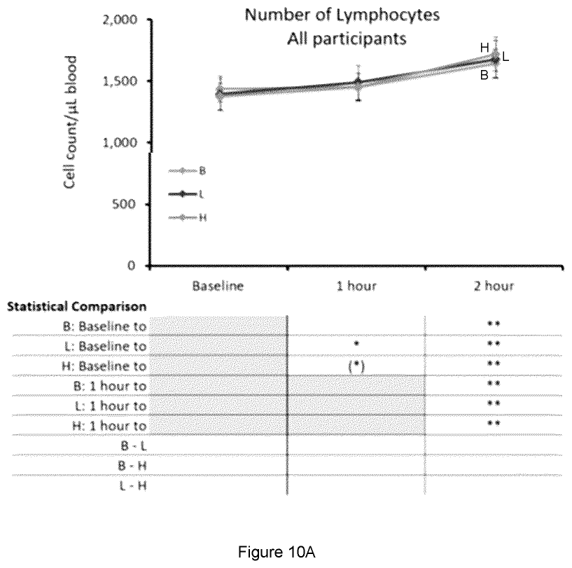

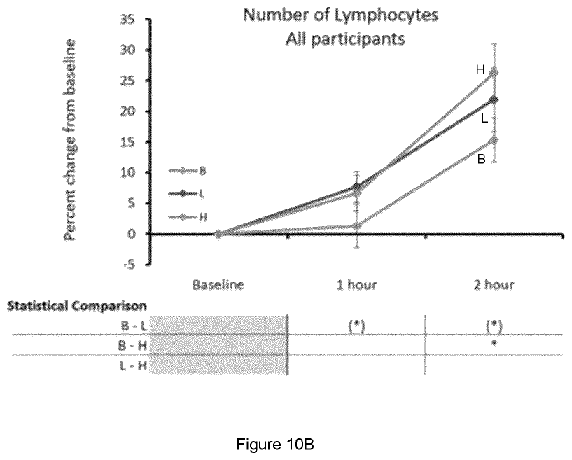

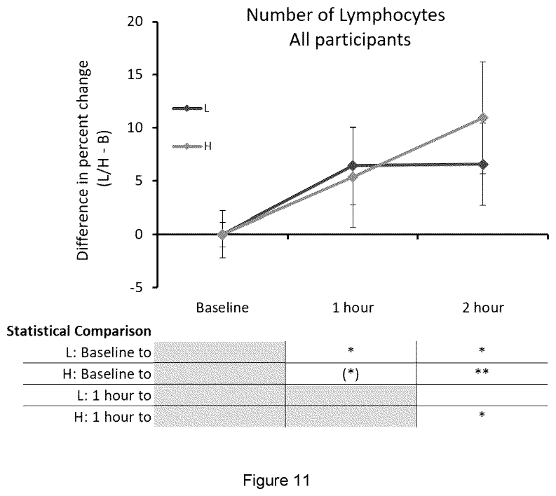

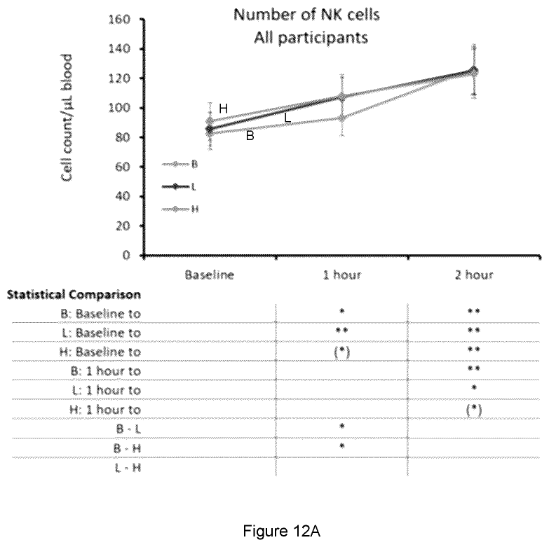

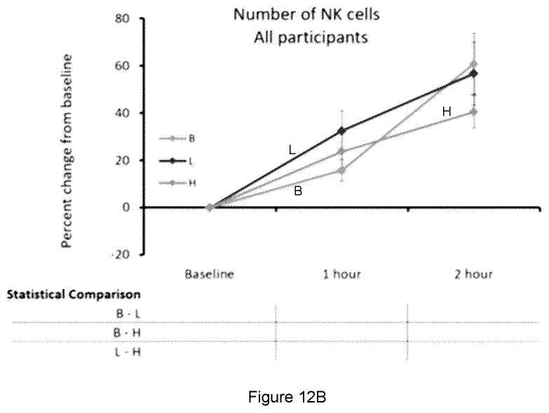

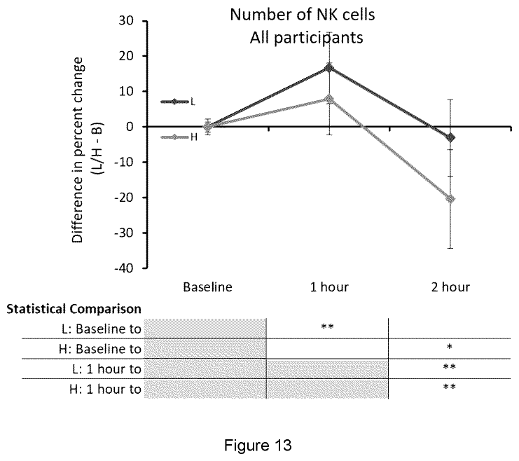

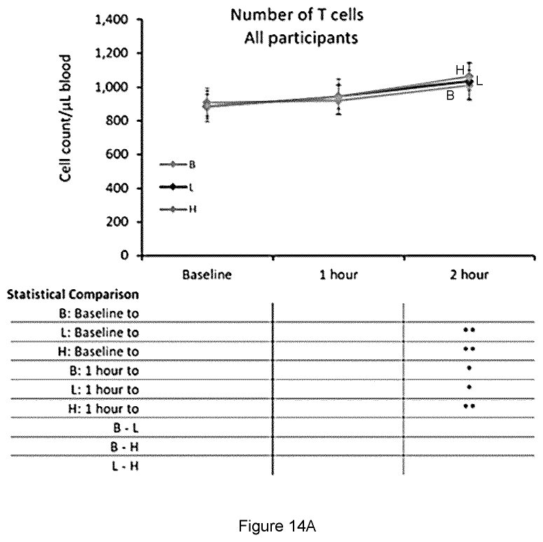

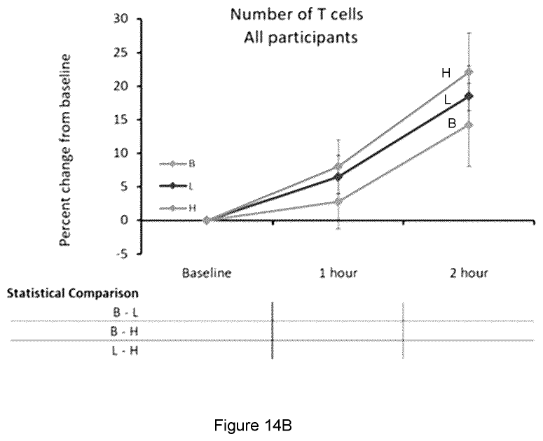

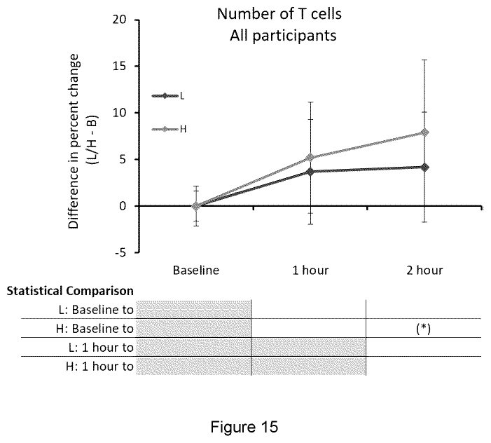

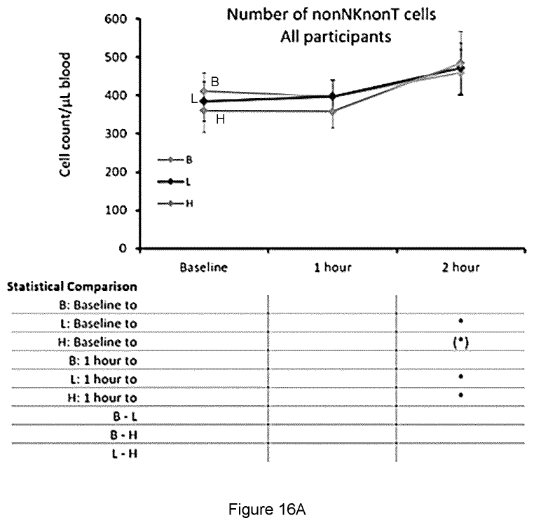

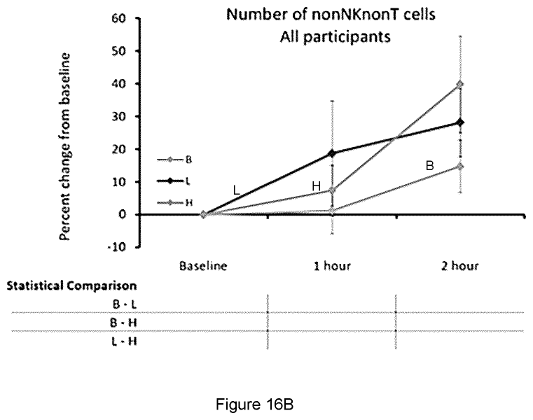

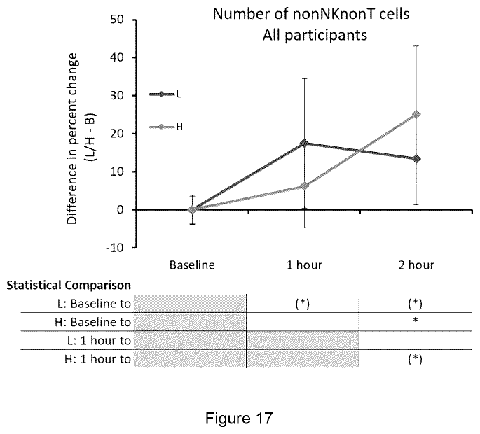

With reference to FIGS. 8-17, consuming the polyphenol seaweed extract powder was associated with increased numbers of immune cells in the blood circulation, providing evidence of mobilization of immune cells.

The low dose of polyphenol seaweed extract powder was associated with:
A 10% increase in average monocyte numbers at 1 hours by the low dose of polyphenol seaweed extract powder was highly significant (P<0.01);
A 6% increase in average lymphocyte numbers was statistically significant at 1 and 2 hours after consumption;
A 17% increase in NK cells at 1 hour after consumption was highly significant; the effect was rapid and transient, and returned to similar levels as placebo at 2 hours;
A mild increase in T cell numbers was seen at 1 and 2 hours after consuming polyphenol seaweed extract powder;
An increase in non-NK non-T cells was seen at 1 and 2 hours, reaching a statistical trend for both time points.

The high dose of polyphenol seaweed extract powder was associated with:
An increase in monocyte numbers at 2 hours after consuming the high dose of polyphenol seaweed extract powder (not statistically significant);
An increase in lymphocyte numbers reached a statistical trend at 1 hour, and a high level of statistical significance at 2 hours after consumption;
A mild increase was followed by a 20% decrease in NK numbers at 2 hours, suggesting homing of the NK cells after consuming polyphenol seaweed extract powder; the decrease at 2 hours was statistically significant.
A mild, 8% increase in T cell numbers reached a statistical trend at 2 hours after consumption;
A gradual increase in the numbers of non-NK non-T cells reached 25% after 2 hours, and this was statistically significant.

An increase in circulating NK cells reached a high level of statistical significance for the lower dose, in contrast to a mild increase for the higher dose at 1 hour, followed by statistically significant decrease of NK cells after consuming the higher dose. The data suggests that consuming polyphenol seaweed extract powder initially triggers mobilization of NK cells (seen for both doses), followed by homing of NK cells. The NK cell homing after consuming the lower dose may take longer time and was not evident at 2 hours after consumption. This is supported by a further observation that after consuming the lower dose, NK cells showed an increase in the expression of the activation marker CD25. In contrast, no increase in CD25 was seen after consuming the higher dose. This, in combination with the increased homing of NK cells after consuming the higher dose, suggests that both doses activate the NK cells, but for the lower dose it takes longer for the activated NK cells to migrate out of the blood stream.

### Stem Cell Effects

Consuming polyphenol seaweed extract powder was associated with selective effects on specific types of stem cells.

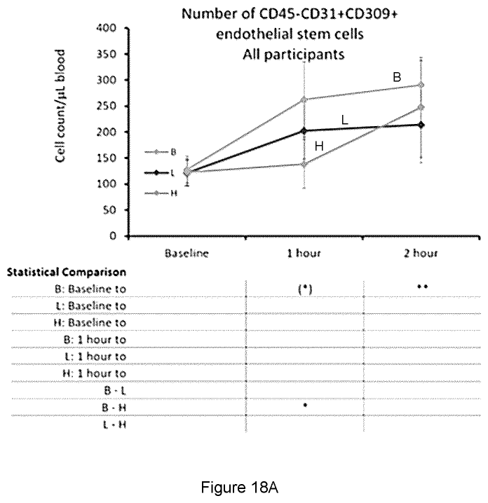

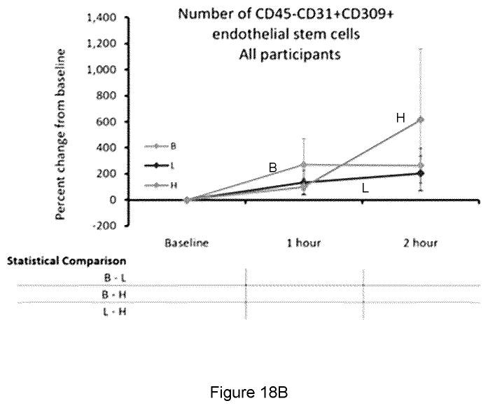

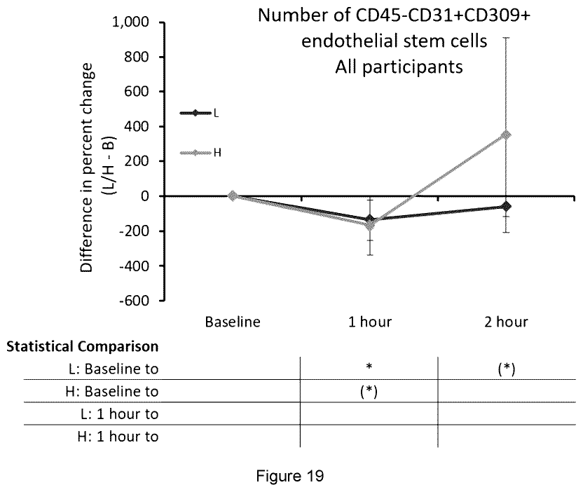

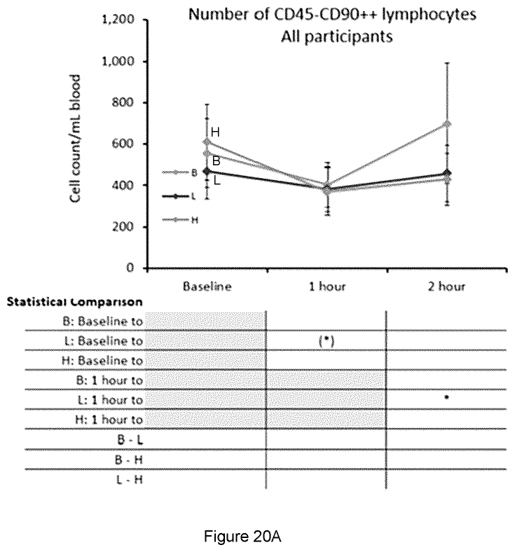

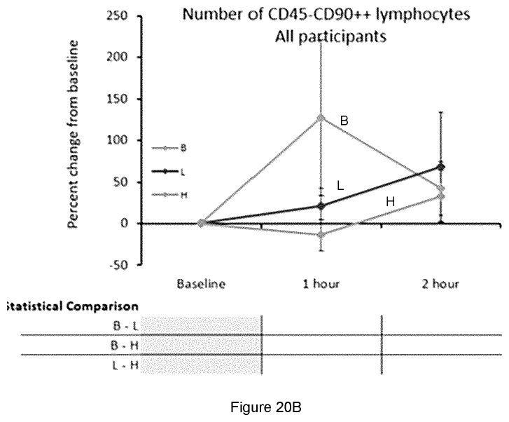

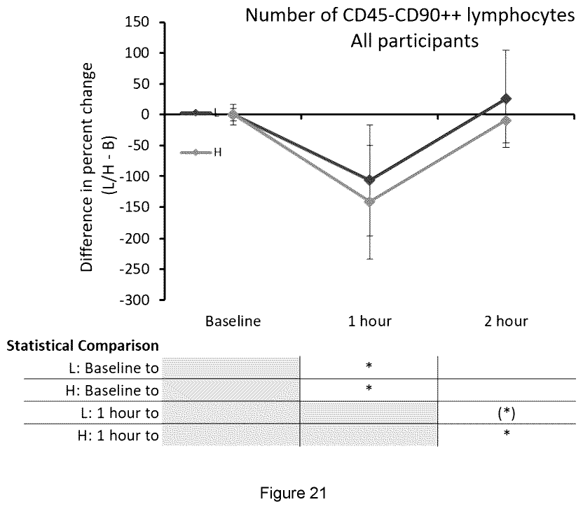

With reference to FIGS. 18-21, there was decreased numbers of two types of stem cells, suggesting homing of these cells into tissues: CD45− CD31+CD309+ endothelial stem cells involved in cardiovascular repair and prevention; and CD45− CD34− CD90+ stem cells, where the phenotype suggests a possible classification as mesenchymal multipotent stem cells. A rapid and transient decrease in average numbers of CD45−CD31+CD309+ endothelial stem cells was seen. At 1 hour after consuming polyphenol seaweed extract powder, the decrease was of a similar magnitude for both doses of polyphenol seaweed extract powder. A rapid and transient decrease in average numbers of CD45−CD90+ lymphocytes was also seen at 1 hour after consumption for both the low and the high doses of the polyphenol seaweed extract powder.

Consuming polyphenol seaweed extract powder also triggered changes to the numbers of Very Small Embryonic-Like stem cells (VSELs). The higher dose produced a faster response than the lower dose, but the increase was of a similar magnitude for both doses. After consuming the high dose of polyphenol seaweed extract powder, a rapid increase in VSELs was seen at 1 hour, but the changes did not reach statistical significance when compared to placebo; the change was bordering a statistical trend (P<0.12). After consuming the low dose of polyphenol seaweed extract powder, an increase in VSELs reached statistical significance at 2 hours.

### Example 4: Fucoidan Production

Fucoidan extraction can suitably performed using seaweed biomass already subject to antioxidant extraction as detailed in Example 1 as detailed below, although it may be performed prior to the antioxidant extraction. References in this example are to percentage of the initial dry seaweed weight used in the antioxidant extraction. References in this example are to percentage of the initial dry seaweed weight used in the antioxidant extraction.

The example of 500 Kg Seaweed is provided, however, batch sizes may vary according to the production needs.

This is a room temperature extraction. Little or no heat is added to the process. Extraction is carried out at 20-30 C.

Tanks, lines and equipment are all thoroughly rinsed with potable water. Seaweed is added to the extraction tank, and it is rinsed with 5 to 1 water for 20 minutes to remove any residue from the antioxidant extraction. The rinse water is discarded, and the seaweed is returned to the tank for extraction.

Water is added to the Extraction Tank. The volume is calculated as 8× the initial dry seaweed weight, i.e. 500 Kg seaweed calls for 4000 L water to be added to the tank.

The organic acid is added, in a total amount of 5% of the original dry seaweed weight, with a goal of achieving a pH of 4.0 to 4.5.

The extraction is performed over a period of 16-24 hours under conditions of gentle agitation.

The Slurry is then pumped over an 80-mesh dewatering screen to separate the solids form the liquids. The seaweed is returned to the hopper and is available for subsequent alginate extraction

The liquor is transferred to a filtering tank. Polyvinylpolypyrrolidone (PVPP) powder is added to the tank as it begins to fill. The amount is calculated as 0.5% of the seaweed weight i.e. for 500 Kg, 2.5 Kg PVPP. The mixture is agitated to keep the solids in suspension. This powder absorbs any residual polyphenols extracted with the fucoidan.

Once the filter tank contains all the liquor, Filter Aid (diatomaceous earth) is added to the tank. The amount is calculated as 7% of the seaweed weight, i.e. for 500 Kg it would be 35 Kg of filter aid.

The Filter press is coated with a thin layer of filter aid. A clean, 1 micron filter bag is placed in the canister after the filter press, as a failsafe, and pressure filtering begins. The clean liquor moves on to the Concentration Tank.

When the liquor begins to fill the Concentration Tank, the Ultra Filter is turned on and concentration begins. The volume of liquid will decrease as water is removed and the Fucoidan becomes more concentrated. Concentration is performed with an ultrafilter. The filter is sized to separate water, salts and unwanted heavy metals while holding back the Fucoidan and in this example, a filter size of 10,000 Daltons is used.

The liquid is concentrated down in volume until a solids content of 10-12%, is reached and transferred to a Finishing tank.

The extract is freeze dried.

The dry product may be ground and an 80 mesh sieve may be used to create a uniform powder.

### Example 5: Alginate Production

In this Example, Alginate extraction is carried out on the same seaweed that was used to product the antioxidant extract (Example 1) and Fucoidan (Example 4). Removal of the polyphenols, dark colour and other polymers, as previously detailed allows for a unique extraction method for alginate; the seaweed does not need to be pretreated or pre-extracted to remove these compounds. References to percentages are to the initial dry seaweed weight used in the antioxidant extraction.

The example of 500 Kg Seaweed is provided, however, batch sizes may vary according to the production needs.

This is a room temperature extraction. Little or no heat is added to the process. Extraction is carried out at 20-30 C.

After the extraction of Fucoidan, the tanks, lines and equipment are all thoroughly rinsed with potable water.

Water is added to the Extraction Tank. The volume is calculated as 8× the initial dry seaweed weight, i.e. 500 Kg seaweed calls for 4000 L water to be added to the tank. The water and seaweed are agitated and Soda Ash (Na₂CO₃) solution is added to the mixture in an amount sufficient to raise the pH to 9.0-9.8.

The mixture is agitated for 16-24 hours.

The Slurry is then mixed with water, to dilute it, before it is pumped over a dewatering screen to separate the solids form the liquids. The seaweed can be collected and suitably used as a soil ingredient or additive.

The liquor is transferred to the filtering tank. Activated carbon powder and PVPP powder are added to the tank as it begins to fill. The amount is calculated as 0.25% of the seaweed weight, i.e. for 500 Kg, 1.25 Kg of each powder. The mixture is agitated to keep the solids in suspension. These powders reduce chemicals that can cause bad odour and/or taste in the final product.

Once the filter tank contains all the liquor, Filter Aid (diatomaceous earth) is added to the tank. The amount is calculated as 7% of the seaweed weight, i.e. for 500 Kg, 35 Kg of filter aid.

The Filter press is coated with a thin layer of filter aid. A clean, 1 micron filter bag is placed in the canister after the filter press, as a failsafe, and pressure filtering begins. The clean liquor moves on to the Concentration Tank.

When the liquor begins to fill the Concentration Tank, the Ultra Filter is turned on and concentration begins.

The volume of liquid decreases as water is removed and the extract becomes more concentrated. Concentration is performed with an ultrafilter. The filter is sized to separate water, salts and unwanted heavy metals while holding back the extract. In this example, a 5,000 Daltons size filter is used.

The liquid is concentrated down in volume until a solids content of 6-8%, is reached and transferred to a Finishing tank.

In a first option, the Alginate extract can be packaged and sold in liquid form.

Alternatively, the extract is freeze dried. The freeze dried product can be ground and an 80 mesh sieve can be used to create a uniform powder.

As a further alternative, the liquid alginate is precipitated to further improve the purity of the final Alginate product. In an exemplary precipitation, the liquid alginate is transferred to a novel precipitator where it is turned to calcium alginate by mixing with a solution of Calcium chloride. The Calcium alginate is separated form the water using a vibrating screen. The solid is then treated with a weak solution of Hydrochloric Acid (HCL) down to pH 1.5 to turn the Calcium alginate to algenic acid.

The algenic acid is mixed with food grade Soda Ash to convert it to water and soluble Sodium Alginate paste. This is dried using hot air in a low temperature dryer. Grinding and sieving can be used to produce a uniform powder.

The purpose of precipitation is to further improve the purity of the final Alginate product.

### REFERENCES

i Jensen G S, Redman K A, Benson K F, Carter S G, Mitzner M A, Reeves S, Robinson L. Antioxidant bioavailability and rapid immune-modulating effects after consumption of a single acute dose of a high-metabolite yeast immunogen: results of a placebo-controlled double-blinded crossover pilot study. J Med Food. 2011 September; 14 (9): 1002-10.
ii Jensen G S, Patel D, Benson K F. A novel extract from bovine colostrum whey supports innate immune functions. II. Rapid changes in cellular immune function in humans. Prev Med. 2012 May; 54 Suppl: S124-9.
iii Jensen G S, Hart A N, Zaske L A, Drapeau C, Gupta N, Schaeffer D J, Cruickshank J A. Mobilization of human CD34+CD133+ and CD34+CD133 (−) stem cells in vivo by consumption of an extract from Aphanizomenon flos-aquae—related to modulation of CXCR4 expression by an L-selectin ligand? Cardiovasc Revasc Med. 2007 July-September; 8 (3): 189-202.
iv Drapeau C, Benson K F, James J, Jensen G S. Aloe macroclada from Madagascar Triggers Transient Bone Marrow Stem Cell Mobilization. J Stem Cell Res Ther 2015, 5:287.

1. A process for extracting compounds from a seaweed biomass comprising:
performing an antioxidant extraction comprising:
a) an aqueous extraction of the seaweed biomass wherein the aqueous extraction is performed in a solvent comprising ≤10% of a C1-C4 alcohol, and wherein the alcohol is used at a volume that is between about 20% and about 40% volume based on the seaweed biomass dry weight, wherein the extraction is performed at a temperature of between about 15° C. and about 60° C. under gentle agitation;
b) separating a crude antioxidant-containing solution from a residual seaweed biomass; and
c) performing ultrafiltration of the crude antioxidant-containing solution to obtain an antioxidant extract containing at least 5% polyphenols by weight.

2. The process of claim 1, further comprising drying the antioxidant extract to obtain an antioxidant powder.

3. The process of claim 2, comprising freeze drying the antioxidant extract to obtain the antioxidant powder.

4. The process of claim 3, wherein prior to freeze drying, the antioxidant extract is frozen over a period of 12 to 48 hours at a temperature of between about −25° C. and about −5° C.

5. The process of claim 1, wherein the crude antioxidant-containing solution is separated from the residual seaweed biomass by filtration or using a centrifugal separator.

6. (canceled)

7. The process of claim 1, further comprising prior to the antioxidant extraction, extracting fucoxanthin from the seaweed biomass using CO₂ supercritical fluid extraction.

8. The process of claim 1, further comprising prior to or following the antioxidant extraction performing a fucoidan extraction using a weak organic acid.

9. The process of claim 1, wherein the seaweed biomass comprises a brown seaweed biomass.

10. The process of claim 1, wherein the seaweed biomass is chopped but not pulverized and dried to a moisture content of ≤about 10%.

11. The process of claim 1, comprising subsequently performing an alginate extraction from the residual biomass, comprising extracting the sodium alginate using a base treatment.

12. The process of claim 11, wherein the sodium alginate extraction further comprises prior to extracting from the residual biomass alginate using a base treatment, treating the residual biomass with a dilute acid.

13. (canceled)

14. The process of claim 1, wherein the seaweed biomass comprises the separated blade and the peeled or removed skin of stype from the genus Laminaria.

15-16. (canceled)

17. Alginate produced by the process of claim 11.

18. (canceled)

19. An antioxidant extract or powder prepared by the process according to claim 1.

20. (canceled)

21. The process of claim 1, wherein the aqueous extraction is performed in a solvent of between about 1% and about 10% of a C1-C4 alcohol and/or wherein the C1-C4 alcohol is ethanol or isopropyl alcohol.

22. The process of claim 21, wherein the aqueous extraction is performed in a solvent of between 2.5% and 4.5% of a C1-C4 alcohol.

23. The process of claim 8 wherein the fucoidan extraction is performed prior to the antioxidant extraction.

24. The process of claim 9, wherein the seaweed biomass comprises brown seaweed biomass from the genus Macrocystis, Laminaria or Ascophyllum.

25. The process of claim 12, wherein the base treatment comprises Na₂CO₃ and/or wherein the dilute acid comprises HCl or MgCl₂.

26. The process of claim 1, wherein the antioxidant extract contains between 5% and 35% polyphenols by weight.
